(19) United States
(12) Patent Application Publication
(10) Pub. No.: US 2006/0228323 A1
(43) Pub. Date: Oct. 12, 2006

(54) Compositions for treating and removing noxious materials malodors and microbes, and methods of use and preparation thereof

(76) Inventors: Andrew Novelle, Auckland (NZ); Roly Crowther, Auckland (NZ); Frank Murphy, Goggenhall Essex (NZ)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 11/095,329

(22) Filed: Mar. 31, 2005

(51) Int. Cl. A61L 9/00

(52) U.S. Cl. 424/76.2

(57) ABSTRACT

A composition for removing noxious or malodorous ingredients from materials containing the same, the composition comprising an amphoteric compound; solvent; a compound selected from the group consisting of acids, salts of the acids and combinations thereof; and water, and wherein the relative amounts of each of the components present is sufficient to effect the desired removal of the noxious or malodorous ingredients. The composition may be used as an antimicrobial, biocide, smoke reduction agent, and is biodegradable.

This application is claims priority from U.S. provisional application No. 60/557,544 filed on march 30, 2004, and all of said provisional application is hereby incorporated by reference.

### FIELD OF THE INVENTION

The present invention relates to chemical formulations and their use in the neutralization and/or deodorization of malodors and toxic gases, and microbial disinfection. The present invention further relates to methods of production of said chemical formulation.

### BACKGROUND OF THE INVENTION

Control of odor and microbial contamination are significant problems for which there are very few adequate solutions. Odor control and removal of noxious and toxic materials such as gases, solids, particulates and liquids gas neutralization are of particular interest in the area of human and animal waste disposal. Generally, disposal of solid waste product from small household animals and pets is accomplished by the use of animal litter that is designed to absorb waste products. However, controlling malodors in the domain of commercial-scale production of animals reaches epic proportions, where tens of thousands of animals may be involved. Such circumstances require an easily-dispensed, environmentally-friendly, and inexpensive solution that would effectively combat the odors and toxic gases produced by animal droppings and other biowaste.

Similarly, inhibition or microbial growth or complete removal of microbes is important in a variety of domestic, healthcare, public heath and industrial situations.

Numerous conventional methods and formulations for eliminating undesirable odors involve the use of fragrant materials to mask the malodors. However, this approach does not result in the elimination of the odors and may actually promote the foul odors.

While several previous approaches seek to eliminate the malodors, they do so via the use of complex formulations that includes enzymes, bacteria, or transition metal complexes. Such formulations are often expensive and may require special handling to be effective.

### SUMMARY OF THE INVENTION

There has been a long standing need for a formulation and method for the neutralization and/or deodorization of malodors and toxic gases. Accordingly, one aspect of the present invention encompasses odor neutralizing formulations. In particular, the present invention relates to a composition and methods of its use that are suitable for eliminating or substantially reducing undesirable odors and toxic gases resulting from animal waste and industrial emissions.

Further, the present invention relates to methods of synthesizing and using said formulations.

More particularly, these advantages and properties are achieved by a composition which comprises an amphoteric compound, and optionally, one or both of an acid and a salt.

Additionally, modification of the basic formulation of the present invention can provide compositions which exhibit other useful properties, for example, antiseptic, antimicrobial properties, e.g. antibacterial, antiviral, antifungal, anti spore, e.g., anti anthrax properties, properties.

The inventive composition is a readily biodegradable, non-staining, non-corrosive, non-flammable and chemically stable. It will not harm humans, animals, minerals or vegetables

Methods of making and using the inventive composition and tests in connection therewith are detailed below.

### BRIEF DESCRIPTION OF THE DRAWINGS

FIG. 1 is a graph of the rate of NH₃ removal using the inventive composition;

FIG. 2 is a graph of the rate of SO₂ removal using the inventive composition;

FIG. 3 is a graph of the rate of H₂S removal using the inventive composition;

FIG. 4 is a graph of the rate of CO₂ removal using the inventive composition; and

FIG. 5 is a graph of the rate of CO removal using the inventive composition.

### DETAILED DESCRIPTION OF THE INVENTION

Gaseous Phase Application

In atmospheric application, the inventive composition concentrate is first mixed with water to a prescribed dilution ratio appropriate to the given application. It is then sprayed or fogged into the atmosphere, at the source or close to, the point of emissions of the toxic and noxious gas. A standard starting point for the amount of the inventive composition to be used is 0.002 g of the inventive composition concentrate per cubic meter of air or 2 g the inventive composition per 1000 m³ of air.

The inventive composition can be used to “blanket” enclosed spaces such as confined animal feeding operations (CAFO) for close to total elimination or to “curtain” either closed or open areas so that the toxic and noxious material cannot pass, such as in rendering plants.

When sprayed or fogged into the atmosphere the inventive composition instantly forms a chemical complex with the gas molecules. On a molecular level, it is essential that the targeted gas molecules contact the micro-droplets of the inventive composition. As mentioned, laboratory tests have shown that the reaction time is instant and occurs in less than 0.1 of a second. Prolonged contact time rapidly accelerates the rate of natural degradation of the gas molecules.

Flocculation

When the inventive composition is applied to liquids that contain high levels of suspended solids, such as slurry pits, oxidation ponds and sewage treatment plants, the inventive composition dramatically reduces the offensive odors and improves flocculation and floc stability. Improvement in flocculation by as much as four times has been reported with a four fold increase in the amount of floe recovered at greatly increased density.

Odors

Most products purporting to eliminate odors are merely “masking agents” seeking to cover odors with a strong smelling perfume, others are simple acidic solutions which work on some types of odor but not on others.

The majority of offensive odors fall into three broad categories:

The “acid” gases, such as hydrogen sulfide and the other sulfur containing compounds are generally classified as mercaptans, with a pH of <7.

The “alkaline” gases, such as ammonia and amine containing compounds.

Generally, these are odors generated from decomposition/putrefaction, with a pH of >7.

The final group contains aldehydes and ketones with little or no pH effect. Many compounds of this group are associated with pleasant smells and are commonly found in industrial fragrances and perfumes used in personal care products.

Many compounds in this group are associated with allergic reactions. Sensitivity to allergens is becoming a major and increasing problem in the industrialized world. Several sources have been identified.
1. Outgassing of building materials, paints, plastics, wall linings, building insulation and electronic equipment.
2. Components of fragrances for personal use and in personal care products such as soaps, shampoos and personal deodorants.
3. Plant pollen and mold and yeast spores
4. Insect detritus such as dust mite feces

Many of the allergens produced in 1 and 2 above are aldehydes and ketones.

Testing has shown that the inventive composition is able to remove odors in the classes described above and more specifically as listed below and following.

TABLE 1
Sulfur Containing Compounds

| | | |
|---|---|---|
| Dimethyl Sulfide | (CH₃)₂S | Decayed vegetable |
| Diphenyl Sulfide | (C₆H₅)₂S | Unpleasant |
| Hydrogen Sulfide | H₂S | Rotten eggs |
| Sulfur Dioxide | S02 | Pungent, irritating |

TABLE 2
Amine Containing Compounds

| | | |
|---|---|---|
| Ammonia | NH₃ | sharp, pungent |

Taurine falls into both the above categories having an acidic sulfur containing active group as well as an amine active group.

Cholic acid is as the name implies acidic but contains neither sulfur nor amine groups.

Both taurine and cholic acid are derivatives of bile used as a precursor in the manufacture of pharmaceuticals.

TABLE 3
Aldehyde Containing Compounds

| | | |
|---|---|---|
| Cinnamaldehyde | (C₆Ii₅)C₃H₃0 | Spicy |
| Formaldehyde | HCHO | Acrid, choking |

Note:
There are many more aldehydes and ketones. These being only the ones tested.

It is the ability of the inventive composition to perform simultaneously at both ends of the pH scale that makes the inventive composition chemically unusual. The inventive composition is effective at eliminating chemical compounds registering<7 acidic, (7=neutral) and >7 alkaline.

The inventive composition reaction with acid rain chemicals.

Laboratory and field tests show the inventive composition is effective in reducing common air pollutants and extremely fine particulates caused by motor vehicles, agricultural traffic and industrial sources. As a result, the inventive composition is particularly effective in removing the broad spectrum of gases that cause acid rain.

TABLE 4
Acid Rain and Green-house Effect Compounds

| | | |
|---|---|---|
| Hydrogen Sulfide | H₂S | total elimination |
| Sulfur Dioxide | SO₂ | total elimination |
| Carbon Dioxide | CO₂ | >70% elimination |
| Carbon Monoxide | CO | >66% elimination |
| Nitrous Oxide | NO | >35% elimination |

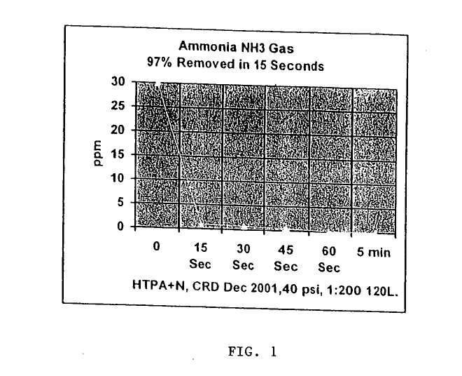

As shown in FIG. 1, the inventive composition removed 97% of NH₃ in 15 seconds and 100% in less than 5 minutes.

Sulfur Dioxide (SO₂) Gas

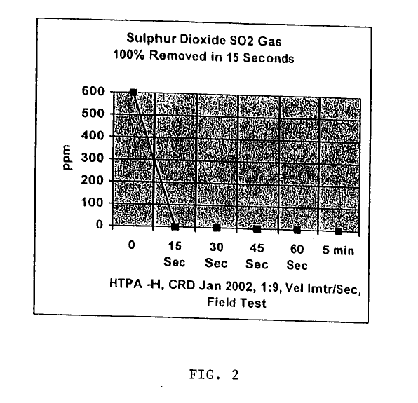

As shown in FIG. 2, the inventive composition removes 100% of sulfur dioxide in less than 15 seconds. It is probable 100% was removed instantaneously on contact.

Hydrogen sulfide is a toxic gas that is heavier than air. At low concentration, it has a characteristic odor similar to rotten eggs. It is produced during anaerobic decomposition of manure and is therefore associated with deep pits such as found in pork production and sewerage treatment plants.

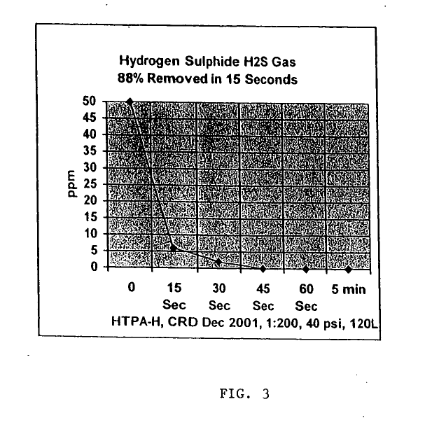

The recommended CAFO level is <10 ppm. As shown in FIG. 3, the inventive composition removed 88% of hydrogen sulfide in les than 15 seconds with 100% elimination achieved in less than 45 seconds.

Carbon dioxide is a known “green-house” gas. The green-house effect is caused by high concentrations of carbon dioxide and other pollutants in the upper atmosphere which act as “heat blanket” by not allowing excess heat from earth to escape into space.

The inventive composition effectively and significantly reduces levels of carbon dioxide.

Levels of 5000 to 15,000 ppm of carbon dioxide have been detected in these sheds. The recommended maximum levels are <2500 ppm.

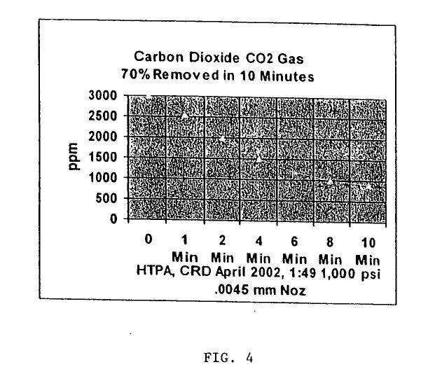

As shown in FIG. 4, the inventive composition removed 70% of the CO₂ in 10 minutes.

Carbon monoxide is a particularly lethal gas even at low concentrations. It is the predominant poisonous gas emitted from gasoline combustion engines.

Carbon monoxide could be a problem in situations where incomplete combustion is occurring in unvented propane or natural gas heaters used in poultry and pig sheds.

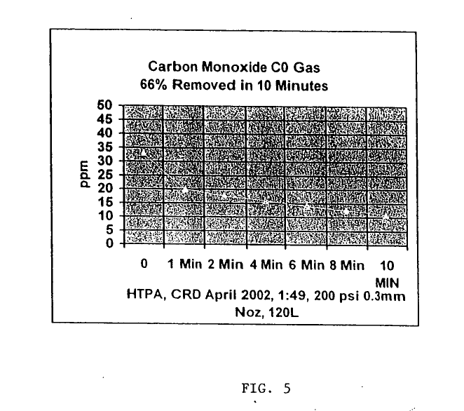

As shown in FIG. 5, the inventive composition removed 66% of the CO gas in 10 minutes.

Generally, the present invention relates to compositions and methods of their use and production that are effective in eliminating or substantially reducing and/or eliminating malodors and toxic gases.

The present invention relates to concentrated odor eliminator for use in a diluted form, effective on both acid and alkaline odors. This concentrated odor eliminator is especially effective on animal or organic sourced odors.

The present invention also broadly relates to a neutralizing composition comprising an amphoteric compound, an organic alcohol, and optionally one or both of an acid and a salt.

The formulations of the present invention preferably include an amphoteric compound, which is preferably an amphoteric surfactant. The amphoteric compound may be for example a linear long chain aliphatic acid with alkyl side chains or long chain fatty acids. Examples of such compounds include alkylbetaines, amidopropyl betaines, amidopropyl sultaines, and combinations thereof. A particularly preferred amphoteric compound for use in the present invention is cocamidopropyl betaine. Furthermore, included in the formulation is preferably an organic alcohol. Narrow and wide cut alcohols with side chains may be used within the context of the present invention. Thus, primary, secondary, or ethoxy alcohols or salts thereof may be employed. For example, sodium lauryl ethoxysulfate may be included into the formulation as an alcohol, even while it is traditionally considered an anionic surfactant.

The formulation may also include an organic acid, desirably a hydroxy acid. Examples of hydroxy acids include citric acid, maleic acid, succinic acid, propenoic acid, glycolic acid, acetic acid, lactic acid, and tartaric acid. A salt is also preferably included in the formulation in the preferred embodiment of the invention which constitutes a salt of an organic acid. Preferably, the salt used is a salt of the hydroxy acid that is also present in the formulation. In a particularly-preferred embodiment, sodium citrate and citric acid may be included in the formulations of the present invention.

In a presently-preferred embodiment of the present invention, the formulation specifically includes an admixture of active ingredients including cocamidopropyl betaine, sodium lauryl ethoxysulfate, citric acid, and sodium citrate provided in an aqueous carrier or diluent such as water.

Preferably, cocamidopropyl betaine is present in a concentration between about 10% and about 21% by weight. Cocamidopropyl betaine as the active ingredient is present in NEOTAINE CLG Iin an amount of about 30% by weight

Preferably, sodium lauryl ethoxysulfate (as Neodol 25-3S.27) is present in a concentration between about 10% and about 40% by weight. The concentration of the sodium lauryl ethoxysulfate in Neodol 25-3S.27 is about 26% by weight.

Preferably, citric acid is present in a concentration between about 0.1% and about 5% by weight.

Preferably, sodium citrate is provided in a concentration range between about 0.1% and about 5% by weight.

Preferably, the balance of the formulation is water (approximately 22.5% by weight).

The cocamidopropyl betaine is present in the composition in the range from about 10 to 25% by weight. The NEOTAINE CLG may for example be present in the currently-preferred formulations at a concentration between about 30% to about 70% by weight. The concentration of the active ingredient in NEODOL 25-3S/27 is around 26% by weight. The concentration of NEODOL 25-3S/27 in the formulations of the present invention may be between about 10% and about 40% by weight.

A presently-preferred embodiment of the formulations of the present invention may be found in Table 1 below.

TABLE 5
BASIC COMPOSITION #1

| Chemical component | Concentration (% by weight) |
|---|---|
| Cocamidopropyl betaine* | 50% |
| *Sodium lauryl ethoxysulfate | 25% |
| Citric acid - anhydrous | 1% |
| Sodium citrate dihydrate | 1.5% |
| Water | 22.5% |

*as Neodaine and Neodol, respectively.

In another presently-preferred embodiment, the formulations of the present invention include an amphoteric compound, an anionic compound, and at least one of an organic alcohol, an acid, a salt, and a preservative. In particularly-preferred embodiments, the acid is a hydroxy acid, the salt is a salt of said acid, and the amphoteric compound is an amphoteric surfactant. In other presently-preferred embodiments, anti microbials and or biocides may be present. As used herein, an antimicrobial means an agent capable of destroying or inhibiting the growth of microorganisms and a biocide means a chemical agent capable of destroying living organisms, e.g., a pesticide.

In some presently-preferred embodiments, the anionic compound is an anionic surfactant. Anionic compounds suitable for use in the present invention are alkyl ether sulfates, acyl isethionates, alkyl ether sulfonates, sarcosinates, sulfosuccinates, taurates, and combinations thereof. A particularly-preferred anionic surfactant is sodium lauryl ethoxysulfate.

Preferably, cocamidopropyl betaine is present in a concentration between about 30% and about 70% by weight.

Preferably, sodium lauryl ethoxysulfate is present in a concentration between about 10% and about 40% by weight.

Preferably, citric acid is present in a concentration between about 0.1% and about 5% by weight.

Preferably, sodium citrate is provided in a concentration range between about 0.1% and about 5% by weight.

Preferably, Bronopol* is provided in a concentration of approximately 0.05% by weight.

Preferably, the balance of the formulation is water (approximately 21.5% by weight).

A particularly preferred embodiment of the present invention is detailed in Table 2A below.

TABLE 6A
BASIC COMPOSITION #2

| Chemical component | Concentration (% by weight) |
|---|---|
| Cocamidopropyl betaine* | 50% |
| Sodium lauryl ethoxysulfate* | 25% |
| Citric acid - anhydrous | 1% |
| Sodium citrate dihydrate | 1.5% |
| Stabilizer | 0.5% |
| Water | 21.5% |

*As Neodaine and Neodol, respectively.

Another particularly preferred embodiment of the present invention is detailed in Table 2B below.

TABLE 6B
BASIC COMPOSITION #2

| Chemical component | Concentration (% by weight) |
|---|---|
| Cocamidopropyl betaine* | 50% |
| Sodium lauryl ethoxysulfate* | 25% |
| Citric acid - anhydrous | 0.5% |
| Sodium citrate dihydrate | 0.75% |
| Stablizer | 0.5% |
| Water | 23.7% |

*As Neodaine and Neodol, respectively.

The formulations of the present invention preferably are effective at eliminating odors over a broad pH range so as to eliminate acidic, neutral, and basic odors.

As used herein, the following abbreviations mean the following:

All figures are expressed as percentages by weight

CAB=Coco amido propyl betaine

SLES=Sodium Lauryl Ethoxy Sulphate

Anti-microbial Bronopol, Myacide Nipacide BIT20 or similar

Citric ac.=citric acid

Sod. Citrate=sodium citrate

Amm. Bifluoride=Ammonium bifluoride

Phosphoric ac.=phosphoric acid 85%

LDAO=Lauryl dimethylamine oxide

Borax decahydrate=Sodium tetra borate decahydrate

MEA=Monoethanolamine

The following tables provide various compositional ranges for the inventive compositions, incluidin preferred compositions:

Bioneutral “Original” Formulation

| | Presently | | | | |
|---|---|---|---|---|---|
| | Preferred | | | | |
| | Embodiment | Range 1 | Range 2 | Range 3 | Range 4 |
| CAB | 15.0% | 5-25 | 10-20 | 13-17 | 14-16 |
| SLES | 6.75 | 0.5-20 | 2-16 | 4-9 | 6-8 |
| Citric ac. | 0.5 | 0.1-2.0 | 0.2-1.3 | 0.3-1.0 | 0.4-0.6 |
| Sod. citrate | 0.75 | 0.2-3.0 | 0.3-1.6 | 0.5-1.5 | 0.6-0.9 |
| Pres. | 0.5 | 0.05-2.5 | 0.1-2.0 | 0.2-1.0 | 0.4-0.6 |
| Water | 76.5 | 94.0-47.0 | 87-59 | 82-70 | 78-73 |

Bioneutral “B” Formulation

| | Presently | | | | |
|---|---|---|---|---|---|
| | Preferred | | | | |
| | Embodiment | Range 1 | Range 2 | Range 3 | Range 4 |
| CAB | 12% | 6-18 | 9-15 | 10.5-13.5 | 11.7-12.3 |
| SLES | 5.4% | 2.7-9 | 3.2-7.6 | 4.5-6.75 | 5.5-5.8 |
| Citric ac. | 0.4% | 0.1-2.0 | 0.2-1.0 | 0.25-0.8 | 0.3-0.5 |
| Amm. | 2.4% | 0.1-5.0 | 0.5-4.0 | 1.0-3.0 | 2.3-2.5 |
| bifluoride | | | | | |
| Phosphoric ac. | 5.0% | 0.2-10 | 1.0-8.0 | 3.0-7.0 | 4.0-6.0 |
| LDAO | 0.4% | 0.1-1.0 | 0.2-0.8 | 0.3-0.6 | 0.35-0.5 |
| Water | 74.4% | 91-55 | 86-64 | 80-68 | 76-72 |
| Triclosan | 2.2% | 0.1-6.0 | 1.0-5.0 | 1.5-3.0 | 2.0-2.5 |

Bioneutral “H” Formulation

| | Presently | | | | |
|---|---|---|---|---|---|
| | Preferred | | | | |
| | Em- | | | | |
| | bodiment | Range 1 | Range 2 | Range 3 | Range 4 |
| CAB | 10.3% | 4.5-16.5 | 6-15 | 7.5-13.5 | 9.6-10.8 |
| SLES | 4.64% | 1.4-12 | 2.7-8.0 | 4-6.75 | 4.3-5.1 |
| Citric ac. | 0.34% | 0.1-3.0 | 0.15-2.0 | 0.2-1.0 | 0.3-0.40 |
| Sod. citrate | 0.51% | 0.15-4.5 | 0.22-3.0 | 0.3-1.5 | 0.45-0.6 |
| Preservative | 0.5% | 0.05-2.5 | 0.1-2.0 | 0.2-1.0 | 0.3-0.6 |
| Borax | 11.9% | 4-16 | 7-15 | 9-14 | 10-13 |
| decahydrate | | | | | |
| MEA | 11.9% | 0.5-35 | 2-25 | 7-14 | 10-13 |
| Water | 59.91% | 89-10 | 82-30 | 72-48 | 65-56.5 |

Bioneutral “N” Formulation

| | Presently | | | | |
|---|---|---|---|---|---|
| | Preferred | | | | |
| | Em- | | | | |
| | bodiment | Range 1 | Range 2 | Range 3 | Range 4 |
| CAB | 13.5% | 9-18% | 10.5-16.5 | 12-15 | 13.2-13.8 |
| SLES | 6.07% | 1.35-10.8% | 3-10.5 | 4-6.5 | 6-7 |
| Citric ac. | 1.45% | 0.1-5% | 0.2-4.0 | 0.5-3.0 | 1.0-2.0 |
| Sod.citrate | 0.675% | 0.1-7.5% | 0.2-5.0 | 0.4-2.0 | 0.5-1.0 |
| Preservative | 0.5% | 0.05-2.5% | 0.1-2.0 | 0.2-1.0 | 0.4-0.6 |
| Water | 77.8% | 89-56% | 86-62 | 83-72 | 78.9-75.6 |

The above formulations can be prepared by the following procedures:
Weigh and mix coco amido propyl betaine & sodium lauryl ethoxy sulphate with gentle agitation to prevent excessive foaming. In cool weather both of these surfactants may need to be conditioned/warmed to permit mixing as when mixed together the resultant becomes very viscous, this is part A.

Seperately mix water citric acid and sodium citrate and preservative, until complete dissolution occurs. This is part B.

Add part B to part A with gentle agitation.

Temperature of ingredients should be in the range 15°-25° C. Preferably 20°.

Temperature:

| Broad | Intermediate | Narrow | Preferred |
|---|---|---|---|
| 10-40° | 15-25° | 18-22° | 20° c. |

As measured by a suitable calibrated thermometer.

pH:

| Broad | Intermediate | Narrow | Preferred |
|---|---|---|---|
| 4-6 | 4.5-6.5 | 4.9-5.1 | 5 |

As measured by a good quality bench model temp/slope compensated pH meter capable of readings to 0.05 pH units.

Viscosity:

Measured at 20° cps Brookfield #3 and #4 spindle

| Broad | Intermediate | Narrow | Preferred |
|---|---|---|---|
| 1000 cps | 1500-6000 c.p.s | 2000-5000 c.p.s | 3500 c.p.s. |
| 8000 c.p.s | | | (centrepoise) |

Mixing:

Low shear, variable speed mixer.

| Broad | Intermediate | Narrow | Preferred |
|---|---|---|---|
| 20-1000 rpm | 40-500 rpm | 50-100 rpm | 60 rpm |

Mixing Time:

Dependent on both temperature of ingredients, viscosity, mix speed, batch size.

| | | | |
|---|---|---|---|
| Broad | Intermediate | Narrow | Preferred |

PART A

| | | | | | | | |
|---|---|---|---|---|---|---|---|
| 1-180 | minutes | 5-120 | minutes | 10-60 | minutes | 30 | minutes |

PART B

| | | | | | | | |
|---|---|---|---|---|---|---|---|
| 0.5-60 | minutes | 5-30 | minutes | 10-20 | minutes | 15 | minutes |

COMBINED PART A & PART B

| | | | | | | | |
|---|---|---|---|---|---|---|---|
| 1-120 | minutes | 10-60 | minutes | 20-40 | minutes | 30 | minutes |

Measured by means of a calibrated electric motor speed controller. For greatest effect and efficiency the smallest droplet/particle size that is consistent with the specific application should be used.

Bioneutral Formula H

PART A

Weigh out and mix together coco amido propyl betaine & sodium lauryl
ethoxy sulphate with gentle stirring.

PART B
Weigh out water and heat to:

| Broad | Intermediate | Narrow | Preferred |
|---|---|---|---|
| 15-95° c. | 35-75° c. | 55-65° c. | 60° c. |

Add sodium tetra borate decahydrate with stirring until completely
dissolved.
Allow to cool to at least 40° c.

PART C

Weigh out sodium citrate, citric acid and water, mix together with stirring.
Add part C to part B with gentle stirring.
Add part B to A & C with gentle stirring.
Mix for 10 minutes.
Weigh out mono elthanolamine and add to the combined mix A + B + C
with stirring.

Observations:
1. If part C is added directly to part A this causes the resultant to thicken dramatically to a viscosity excess of 15000 cps making proper dispersal extremely difficult.
2. By premixing part B and part C and adding this to part A some significant thickening occurs temporarily followed by thinning again.

3. The finished product is when freshly made at 18°-20° C. of a viscosity of 2500-3000 cps, on standing undisturbed over period of days thickens significantly to 4000-7000 cps.

| | | | |
|---|---|---|---|
| Broad | Intermediate | Narrow | Preferred |

Temperature

| | | | | | | | | |
|---|---|---|---|---|---|---|---|---|
| Part A | 10-45° | c. | 15-30° | c. | 18-22° | c. | 20° | c. |
| Part B | 15-75° | c. | 35-75° | c. | 55-65° | c. | 60° | c. |
| Part C | 5-50° | c. | 15-30° | c. | 18-22° | c. | 20° | c. |

Viscosity
Of the undisturbed product at least 7 days after manufacture at 20° c.
As measured by Brookfield viscometer spindle #2, #3, #4

| | | | | | | | |
|---|---|---|---|---|---|---|---|
| 1000-8000 | cps | 3000-6000 | cps | 3500-4500 | cps | 4000 | cps |

pH

| | | | |
|---|---|---|---|
| 7-12 | 8-11 | 9.5-10.5 | 10 |

Mixing times

| | | | | | | | | |
|---|---|---|---|---|---|---|---|---|
| Part A | 5-120 | minutes | 10-60 | minutes | 20-40 | minutes | 30 | minutes |
| Part B | 2-180 | minutes | 20-120 | minutes | 35-45 | minutes | 40 | minutes |
| Part C | 2-30 | minutes | 5-20 | minutes | 10-15 | minutes | 12 | minutes |
| Combined | 5-120 | minutes | 20-60 | minutes | 25-35 | minutes | 30 | minutes |
| mixture | | | | | | | | |

Mixing speeds
Low shear variable speed mixer as determined by calibrated electronic
controller

| | | | | | | | | |
|---|---|---|---|---|---|---|---|---|
| Part A | 5-1000 | rpm | 10-500 | rpm | 20-50 | rpm | 30 | rpm |
| Part B | 5-1000 | rpm | 10-500 | rmp | 20-50 | rpm | 30 | rpm |
| Part C | 5-1000 | rpm | 10-500 | rmp | 20-100 | rpm | 50 | rpm |
| Combined mix | 5-1000 | rpm | 10-500 | rmp | 20-50 | rmp | 30 | rpm |

Bioneutral Formula N
Part A Weigh and mix coco amido propyl betaine and sodium lauryl ethoxy sulphate
Part B Weigh water, sodium citrate, citric acid and mix until dry ingredients are dissolved.

Add Part B to Part A slowly with gentle mixing.

| | Broad | Intermediate | Narrow | Preferred |
|---|---|---|---|---|
| Ph | 2-7 | 3-5 | 3.5.-4.5 | 4.0 |

Mixing, viscosity, temperature parameters as for other formulae.

Bioneutral Formula B

Part A Weigh and mix cco amindo propyl betaine and sodium lauryl ethoxy sulphate

Part B Weigh and mix water, citric acid and preservative

Part C Weigh and mix ammonium bi fluoride and water. When dissolved add to Part C phosphoric acid (85%) and Lauryl dimethyl amine oxide and mix.

Add Part B to Part A with gentle mixing. The mixture will become very thick and gel like.

Continue stirring and slowly add Part C to mix A & B. NB: If addition is not very slow allow consistant mixing the end product will separate.

| | Broad | Intermediate | Narrow | Preferred |
|---|---|---|---|---|
| Ph | 0-7 | 1-5 | 2-4 | 2.8-3.2 |
| Viscosity | 0-4000 cps | 100-3000 cps | 500-2000 cps | 1300-1700 |

All other parameters of preparation as for other formulae.

Method of Use:

Depending on the nature of the substate to be treated, one or more of the formulations may be to treat a target substate which exists in either:

(i) The gas phase or

(ii) The liquid phase or

(iii) To treat a solid which by its porous and/or absorptive nature contains a portion of substate gas and/or liquid phase.

Gas Phase:

In devising a means of treating a targeted substate in the Gas Phase, the following variables must be taken into consideration.

Variables—Velocity of Gas Flow

volume of gas flow
loading of target substate in the gas flow
form of product to be dispersed, spraying or misting or fogging
means by which dispensing will be achieved
dilution of the appropriate formula to be used
means of ensuring mixing of delivered product with the targeted substate i.e. turbulence, induced or otherwise
droplet size of product

Droplet/Particle Size

For the most efficient removal of the targeted substate(s) droplet size is important.

Since droplets of the appropriate formula must contact molecules and or particles of the targeted substate to work, the smaller the droplet size and therefore the greater the number of droplets for any given quantity of product the greater the opportunity to interact with the substate.

However, other considerations such as gas velocities, unpredictable turbulence or external drafts and situation of the target may require either larger than optimum droplets or a means of delivery which will not produce the finest of droplets.

For ease of description, the following definitions apply:

| | | | |
|---|---|---|---|
| Spray | droplet size | 20-80+ | microns |
| Mist | droplet size | 5-20 | microns |
| Fog | droplet size | <5 | microns |

Use in a moving gas stream e.g. chimneys, exhaust outlet, air conditioning, ventilation system:

In a moving gas stream of from 0.5 m/s upwards. The chosen formula is sprayed or misted directly into the gas stream in such a manner as to cover the full cross-sectional area of the gas flow using one of the means described under “Methods of Delivery”, particularly No. 1 or No. 2.

Use in still gas systems e.g. warehouse or open plan building, room or other similar space where the air movement is <0.5 m/s:

In these circumstances, methods of delivery which produce the smallest droplet sizes may be employed by using the appropriate formulation at an appropriate dilution in a delivery system as described in “Methods of Delivery” No's 3, 5, 6, 7, 8, 9 or 10.

Examples of Use in Gas Phase Systems:

1. Reduction/elimination of pollutant gases and particularly emanating from a coal fired process. Such a system would use a delivery system as described under “Methods of Delivery” No's 1 and 2.

2. Suppression and elimination of toxic gases produced in combustion in domestic and commercial accidental fires. Such a system would use a delivery system such as described in 5, 7, 8 of “Methods of Delivery”.

3. Suppression of burnt odours in both building structural materials and smoke damaged retail or commercial goods. Using a method such No. 9 and 10 under “Methods of Delivery”.

Liquid Phase Use:

Use in the liquid phase requires only that

(i) sufficient concentration of the appropriate formulation is present

(ii) that efficient mixing take place

(iii) that in situations where the production of foam would be undesirable that formulation foaming characteristics are suppressed without effecting the formulations desired properties.

Delivery in a liquid system is confined to 1 and 2 under “Methods of Delivery” deffering in that a pressure pump need not be used and that product may be injected directly into the substate liquid without first passing through spraying or misting nozzles.

Examples of Use in Liquid Phase Systems:

1. Treatment of liquid waste streams in sewage plants to abate adour nuisance eg. Municipal sewage utilities

2. Treatment of trade liquid wastes from industrial sites prior to discharge eg. Meat packing plant waste, cannery waste

Use on Solids:

Treatment of solids generally requires the wetting of the surface and so method of delivery is simple.

Such treatment requires coarse sprays and this may be supplied both by manually operated systems such as 4 or 6 under “Methods of Delivery”.

Examples of use on Solids and Surfaces:

1. Spraying of clothing and footwear to abate odour.

2. Spraying of surfaces to eliminate micro organisms.

Methods of Delivery:

Method of delivery of a particular formula for any given application will be chosen from one or more of the following delivery systems.

1. Spray or misting system in which the requisite formula is prediluted and delivered via a liquid pressurizing pump through a suitable filter to the nozzle(s) selected for the particular application. NB: Filter must remove particles smaller than nozzle orifice.

The pressure pump is fitted with a bypass such that product delivered by this pump in excess of the delivery capability of the nozzles is returned to the low pressure side of the pump. This measure prevents foaming and cavitating inside the pump which would result in delivery failure.

The pressure pump selected will be capable of delivering product at pressure of between 50 psi and 1000 psi.

2. Spray or misting system in which a dosing pump capable of increasing or decreasing the amount of product supplied is used. Such a positive displacement pump would be variable in either speed of stroke or length of stoke or both.

The dosing pump feeds the particular concentrated formula into a water supply pipe feeding in turn to a liquid pressure pump such that the formula is introduced to the low pressure side of the pressure pump. Passage of water and formula through the pressure pump ensures thorough mixing. Mixed diluted product then passes through a filter of sufficient fineness, that particles/contaminants of the same size and larger as the nozzle(s) orifice size are removed. The product is then distributed to one or more nozzles as required.

The pressure pump is fitted with a bypass such that product delivered by this pump in excess of the delivery capability of the nozzles is returned to the low pressure side of the pump. This measure prevents foaming and cavitating inside the pump which would result in delivery failure.

The pressure pump selected will be capable of delivering product at pressure of between 50 psi and 1000 psi.

3. A misting or fogging system in which a measured quantity of appropriately diluted chosen formulation is held in a reservoir. A venturi tube leads from the reservoir to an aspirator type stainless steel nozzle. The nozzle is further supplied with compressed air having a pressure of between 50 psi and 500 psi. When activated the compressed air passes through the nozzle and in so doing draws up product from the reservoir by Venturi effect.

The system is surrounded by a metal or plastic shroud open at front and back. Mounted axially in the shroud is a fan normally electrically operated.

Aspirator nozzle with product reservoir and supplied compressed air provide a fine mist or fog, the fan provided disperses the mist or fog throughout large volume of air space.

4. An appropriate formulation at a particular dilution is placed in a bottle, flask or tube fitted with one of (i) a “finger misting pump” or (ii) a “trigger spray pump”.

The “finger misting pump” or “trigger spray pump” is manually activated by the user and aimed directly at the substate or source to be treated.

This delivery method is intended for consumer retail and light commercial markets.

5. Selected formulation placed in a pressurized aerosol can. The aerosol can is placed in or adjacent to an actuating device. The activating device consists of a detector chosen from on of:
(i) optical smoke sensor
(ii) an ionizing smoke sensor
(iii) a carbon monoxide sensor
(iv) a similar noxious or toxic gas or biological threat sensor.

The detector on sensing the particular threat, activates the device which in turn activates the aerosol can.

Once activated the aerosol can remains activated and delivers all of its contents.

6. The appropriate formulation is filled into a pressurized aerosol can. This aerosol can is manually operated and directed. This system is intended for the consumer, retail, light commercial market.

7. The appropriate formulation for the intended application is filled into a device similar to a fire extinguisher. This device may be either of manually activated and directed form or automatically activated by means of a sensor as described in (5) and directed in a fixed path.

8. The appropriate formulation for the intended application may be introduced into a standard commercial or industrial fire sprinkler system. The introduction of the formula is by means of a previously described dosing pump activated by a pressure switch or other suitable device. Such that if the sprinkler system is activated, formulation is pumped into the water reticulation pipes feeding the sprinkler system, the formulation is thus directed to the area of need by the sprinkler system.

9. An ultrasonic fogging system in which the device contains a reservoir of the appropriate formulation at an appropriate dilution. Situated in the reservoir is an ultrasonic transducer. The ultrasonic transducer produces a very fine mist or fog in the air space above the product in the reservoir.

An electrically operated fan is installed in the device. The fan draws the fine mist or fog from the reservoir air space and distributes it into the space to be treated.

The mist or fog so produced is composed of very small droplets. The extremely small droplets thus produced have highly penetrating and excellent dispersal characteristics.

10. The appropriate formulation at an appropriate dilution is placed in the product reservoir of a device known as a thermofogger. Such devices consist of a small “pulse jet” motor powered by some fuel such as gasoline, kerosene, compressed natural gas (CNG) or liquified petroleum gas (LPG).

The “pulse jet” when operated supplies both heat and pressure to vapourize and disperse in fine droplet form the product contained in the reservoir. These devices are built in several sizes and are more or less portable. They may be diluted up to one times 500.

It is contemplated that the formulations of the present invention will have a diverse range of applications in which neutralization of malodors and toxic gases is desired. These applications include use in household lavatories, in enclosed spaces for eliminating smoke fumes, for industrial or agricultural use for eliminating odors, reducing or neutralizing odors from animal droppings, biosolids from sewage, and the like. In addition, the formulations of the present invention may be used in treating fertilizer, mushroom manure, or in deodorizing areas where animals live including barns, dog houses, pet beds, kennels, and zoos. The formulations of the present invention may also be used to treat baby diapers for malodors, as well as gymnasiums, locker rooms, and lavatories. Also the invention compostion may be used to control pet odor.

In addition, the formulations of the present invention possess anti-microbial properties, as shown below. Accordingly, the formulations of the present invention may be used in circumstances in which it is desired to kill [the soectrum of harmful microbes including] bacteria, viruses, yeast and mold fungi. The formulations of the present invention are further useful in killing or retarding the growth of both gram negative and gram positive bacteria. In particular, the formulations of the present invention are useful in kill both the spore and vegetative state of Bacillus anthracis.

The formulations of the present invention are such that they can be sprayed and the spray particles may be directed at gaseous odors. For example, a spray applicator can be fitted in a room and the spray therefrom can be directed at smoke particles. Alternatively, a spray application can be fitted adjacent to the bowl of a toilet and the spray can be directed therein. In yet a further embodiment, the present invention may broadly be comprised of a method of eliminating or substantially removing undesirable odors from a selected area comprising the step of substantially contacting said odors or malodor-producing materials with the formulations of the present invention.

In one presently-preferred embodiment, the formulations may be sprayed towards or formed as an aerosol in the vicinity of the malodors. Alternatively, the formulations may be applied in liquid form to the malodor-producing materials or contacted with the malodorous gases.

Additional uses for the inventive composition include:

Agricultural Products (12)

Broiler House Starter Treatment;

Internal poultry shed environment control;

House Finisher Treatment;

Internal poultry shed environment control day 11

to processing;

Broiler Litter Treatment;

Litter bactericide antiseptic treatment between runs (G)

Broiler External Treatment.

External shed manure and odor treatment;

Layer Hen House Treatment.

Internal poultry shed environment control;

Layer Hen Litter Treatment.

Battery cage litter bactericide treatment;

Layer Hen External Treatment.

External shed odor and manure treatment;

Pig Nursery and Finisher Treatment.

Internal pig shed environment control;

Pig Farm External Treatment.

External oxidation and slurry control;

Cattle Confinement Treatment.

Beef Cattle feedlot shed environment control;

Dairy Farm Effluent and Irrigation Treatment.

Milking shed waste water and pond treatment.

Environmental/Industrial Products

Industrial Smokestack Gas Emission Control; removal of H2S, SO2, CO2, CO, NO (G)

Liquid & Wastewater Treatment.

Oxidation ponds, septic tanks, drains, sludge pits;

Tannery and Pelt Processing.

Hide processing emission control;

Offal, rendering and Meat Processing Plant.

Emission control;

Pulp and Paper Mill Treatment.

External soak and oxidation pond odor control;

Pulp and Paper Mill Treatment, Fiber Cement-Board Toxic Emission Treatment.

Food and Cannery Plant Treatment.

External processing “stic” ponds control;

Food and Cannery Plant Treatment.

Odor Emission Control

Cooking, Boiling wide-spectrum of odors;

High Volume Pedestrian Precincts

Bactericidal, Germ and Toxin Removal

Air Conditioning Bactericidal Treatment.

Hospitals, commercial & residential buildings removing harmful bacterial and nosocomial pathogens.

Application to fabrics and filter material to render them microbe-free and to assist in removal of chemicals, microbes and particulate matter.

Food Processing Plant Hygiene Treatment.

Removal of Bacteria, Listeria in particular

Border Biosecurity Control

Fumigation and Decontamination at Airports & Seaports

(Both craft and shipping containers)

Subways Air Reticulation Treatment.

Removal of Carbon Particulates CO2, H2S, CO

Passenger Aircraft Air-conditioning Bactericidal Germ Treatment. Including public vehicle (buses, trains) transport

Surface Hygiene Treatment.

Commercial use in hospitals for the elimination of bacteria contaminated surfaces, equipment and facilities
Port-a-Loo Bactericidal Treatment
Removal of bacteria and odor
Building Mold Removal Commercial/Consumer
Building Mold and Yeast Elimination.
Commercial applicators for residential and commercial properties covering mold (fungi) treatment internal timber framing, internal services, cladding and insurance work (“leaky building”)
Building Mold and Yeast Elimination.
Consumer product for home use
Carpet Mold Prevention and Removal.
Specific carpet treatment for commercial applicators
Carpet Mold Prevention and Removal.
Consumer product for home use
Wallboard preparation mold treatment.
Pre-treatment prior to wallpapering and painting (acrylic) to prevent mold occurring
Medical and Personal Health Care Products
These include Human Medical/Surgical topical antiseptic/disinfectant uses, Medical/Surgical equipment sterilant and disinfectant uses, Animal Medical/Surgical antiseptic/disinfectant/sterilant uses, human topical wound/sore/ulcer disinfectant/antibacterial applications, human topical fungicide e.g. under toe and finger nail infections; disinfectant soap solutions, pre-Op Surgical Scrub solution, and surgical Instrument sterilant (as opposed to a disinfectant)
Bactericidal Surgical Face Masks and Disposable Garments.
   Masks, headgear, footwear and disposable cloak bacterial protection.
Antimicrobial and Infection Protection Masks.
   Antimicrobial and odor protective face masks specifically packaged for emergency services; ambulance, paramedics, fire-services, mortuaries, funeral homes and police services.
   Fire Smoke Inhalation Mask.
Face mask to reduce smoke inhalation from house fires and high-rise tenants.
Tampons (prevention of Toxic Shock Syndrome), Sanitary Pads.
Age-Care & Living Environment.
Deodorizing spray applicator for removal of ‘living’ odors and protection against potential bacterial infection in rest homes and geriatric care/hospitals.
Bacterial & Odor Elimination Ostomy Products. Removal of unpleasant odor and bacterial infection for stoma bags and incontinence garments.
Spray pack for personal use in changing of ostomy garments.
Anti-Biological-Chemical Terrorist/Warfare Products (namely anthrax)
Commercial Building Air Conditioning Systems.
Commercial applicators product.
Rechargeable Gas Bottle Extinguisher.
For commercial offices, large residential complexes and homes.
Consumer Aerosol Canister.
For home use and the creation of a “Safe-room”.
Biological & Chemical Attack/Warfare Surface Decontamination Wash-down Product.
Commercial liquid spray and wash-down application post attack.
Commercial and Consumer (FMCG) Products
   RES Hospital & Rest Home Incontinence Odor Treatment.
   Additive to Acrylic Paint Odor Removal.
   Odor Prevention and Air Purifying Spray and Aerosol.
   Liquid Toilet Rim Sanitizer.
   Disinfectant wipes.
   Foot Odor-Treatment of Shoe Insoles.
   Furniture Fabric, Drapes & Carpet Freshener
Boat and Recreational Craft (septic holding tank treatment.
Marine toilet/heads pre-conditioning.
   Refrigerator air cleanser and bacterial decontamination.
   Pet Goods
   Veterinary clinic antibacterial surface treatment.
   Odor prevention and elimonation for kennels and bedding
   Kitty litter Odor Eliminator
   Odor Eliminator and Prevention Spray for Domestic Animals
   Commercial/Hospitality/Dry Cleaning Products
   Hotel, Restaurant, Bar & Casino Air Conditioning Treatment.
   Elimination of cigarette and cigar smoke, odor and particulates of commercial premises.
   House Brand Aerosol, Air Conditioning Manufacturers, Installers and Service Operators. (e.g. Carrier, Scope, Mitsubishi, etc.)
   Insurance Dry Cleaning Product.
   Removal of smoke odor from fire smoke damaged garments
   Consumer Aerosol Spray.
   Elimination of cigarette and cigar smoke odor.
   Air Purification Unit.
   A stand-alone air purifying unit that circulates the room air through the inventive composition-S* filters. Removing smoke odors, particulates and pathogens. Units refilled by proprietary dispensers
   Fire Remediation uses

The inventive composition may be used to remove the toxic gases, noxious odors as well as soot resulting from home and industrial fires. It may also be mixed with fire sprinkler water to alleviate and remove odor resulting from fire extinguishing.

The inventive compositions are useful for introduction into water to be released fire extinguishing sprinkler systems for eliminating odor, asphyxiating gasses and removal of particulate matter in smoke.

The inventive composition may also be incorporated operation with a fire detection system such that upon detection of a fire, the composition is released into the area of the fire to neutralize the toxic gas, remove or alleviate the odor caused by the fire and smoke.

The composition thus formed may be used in the concentrated form or can be further diluted with water depending on the final application. The preferred method of manufacturing the composition involves mixing approximately 50 grams of cocamidopropyl betaine with approximately 25 grams of lauryl ethoxysulfate. Citric acid (approximately 1 gram) and sodium citrate (approximately 1.5 grams) are added to approximately 21.5 milliliters of water so as to allow the citric acid and sodium citrate to dissolve. The aqueous solution is then added slowly to the admixture of cocamidopropyl betaine and lauryl ethoxysulfate with slow stirring. Approximately one gram of bronopol may then be added as a preservative. Care must be taken during mixing of the formulation to avoid creating a solution that is too viscous. Inappropriate stirring or adding the aqueous solution too rapidly could result in the formation of a viscous, ropey solution. This example may be scaled up or down depending on the volume of solution required for the application at hand.

Another preferred method of manufacturing the composition involves measuring the cocamidopropyl betaine out to 50% and heating it to about 25° C., so that the final temperature when finished is more than 15° C. While the cocamidopropyl betaine is stirred, the sodium lauryl ethoxysulfate is measured to 25% and slowly added. This mixture is then mixed for about 15 minutes.

If desired, the viscosity of the concentrate can be controlled by varying the salt/glycerol content, inherent in the manufacture of cocamidopropyl betaine. More salt, less glycerol results in higher viscosity. This can also be achieved by including a small (typically 25%) proportion of CAB GF (glycerol free) in the total CAB content.

Then 0.75% of sodium citrate, 0.5% of citric acid and 0.05% of bronopol is added to about 4.5% of warm pure filtered water. This mixture is agitated until the citric acid and sodium citrate is completely dissolved in the water.

The mixture of cocamidopropyl betaine and sodium lauryl ethoxysulfate is stirred mildly, and these dissolved powders are added. The viscosity will increase marginally. This mixture is then mixed for about 15 minutes.

Alternatively, for more effectiveness with acidic odors, the level of citric acid is lowered or about 0.1% monoethanolamine is added to the final product.

For more effectiveness with alkaline odors, the level of sodium citrate is lowered, or the level of citric acid is increased, up t+/−100% of the standard ingredient quantity. This can result in a significant viscosity increase, depending on the degree of change.

Using pure filtered water, the total quantity is made up to the prescribed volume. This may be warmed in winter to keep the finished product above 15° C. Optional ingredients are often included in the water. This product should be left at least one day, at above 15° C. to complete its combination and de-aeration.

Optionally, fragrance may be added to this product. The fragrance level is generally about 1.5%. To this is added 0.75% of QAC, which is thoroughly mixed at slow speed so as not to ‘bruise’ the fragrance. This mixture will be initially be cloudy but will become clear if left to stand for several hours.

This mixture is then added to the product, and mixed gently but quickly and thoroughly, so as to complete as much of the mixing as possible before the product thickens. Once the product has thickened due to the fragrance, further mixing must be done carefully, or else permanent emulsification will take place, giving the product a whitish appearance. Tests indicate that the product is still effective in this state. Incomplete mixing will result in zones within the mixture being above the 2% concentration level, and some ‘dropout’ may result. This ‘dropout’ should be stirred into the concentrate just before dilution.

The composition so formed can be used in the concentrate form, or can be diluted with pure filtered water, in dilution rates from 9 to one, up to 2000 to one, depending on the final application.

The utility of the present invention may be expanded by the addition of various components to the basic composition described above. These include:

A. A composition for use in destroying odor-producing alkaline substances, e.g., ammonia, either in the gaseous or liquid state.

| | |
|---|---|
| Basic composition #2(Table 2) | 90% |
| Citric acid (10% solution) | 10% |

The level of citric acid may be adjusted to suit the particular situation.

B. This composition exhibits antibacterial properties and is particularly in conjunction with the destruction of alkaline substances.

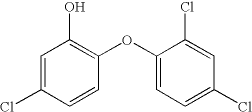

| | |
|---|---|
| Triclosan | 2% |
| Composition A (above) | 98% |

C. This composition is useful to destroy odor producing compounds which are acidic in nature, e.g., hydrogen sulfide, sulfur dioxide, carbon dioxide, and the like.

| | |
|---|---|
| Basic composition #2 | 68.0% |
| Sodium tetraborate (decahydrate) | 12.5% |
| Monoethanolamine | 12.0% |
| H₂O (90° C.+) | 7.5% |

D. This composition is for use in destroying odor-producing acidic substances, e.g., hydrogen sulfide, sulfur dioxide, carbon dioxide, and the like, and when enhanced antibacterial activity is required.

| | |
|---|---|
| Composition C (above) | 90% |
| Triclosan | 2% |

This composition exhibits more effective antimicrobial properties than either the Basic composition or Composition B.

E. This variant is specifically designed to break the amide bonds of the outer coat of Bacillus anthracis spores. This is achieved firstly because halogen acids cause cleavage of these bonds and secondly because this halogen acid is small enough to have a good chance of penetrating the “forest” of side chains of the coating which stick up from the surface and to a large degree must be responsible for its chemical resistance by physically keeping potential attacking species away from the surface proper.

However for the same reason that it carries out the above function it will also disrupt proteins and similar substances such as the capsule of viruses and the large molecule toxins such as Botulin and Ricin

First Component:

| | |
|---|---|
| Basic composition #2 (without sodium citrate) | 80% |
| Second Component: | 20% |
| Composed of: | |
| Water | 61% |
| Ammonium bifluoride | 12% |
| Phosphoric acid | 25% |
| Lauryl dimethylamino oxide | 2% |
| Composition F | |
| This composition exhibits enhanced antimicrobial activity. | |
| Composition E | 98% |
| Triclosan | 2% |

The formulations of the present invention are also effective at inhibiting the growth of various microbiological organisms that are harmful to human health. The effects of formulations of the present invention on several microorganisms were evaluated.

1. A composition for removing noxious or malodorous ingredients from materials containing the same, the composition comprising the following components:
an amphoteric compound;
an organic solvent;
a compound selected from the group consisting of acids, salts of the acids and combinations thereof; and
water, and wherein
the relative amounts of each of the components present is sufficient to effect the desired removal of the noxious or malodorous ingredients.

2. The composition of claim 1, wherein the amphoteric compound is an amphoteric surfactant.

3. The composition of claim 1, wherein the amphoteric compound is a linear long chain aliphatic acid with alkyl side chains or long chain fatty acids.

4. The composition of claim 1, wherein the amphoteric compound is selected from the group consisting of alkylbetaines, amidopropyl betaines, amidopropyl sultaines and combinations thereof.

5. The composition of claim 1, wherein the amphoteric compound is cocamidopropyl betaine.

6. The composition of claim 3, wherein the cocamidopropyl dimethylbetaine is present in an amount of from about 5.0% to about 25% by weight.

7. The composition of claim 6, wherein sodium lauryl ethoxysulfate is present in a concentration of from about 0.05% to about 20% by weight.

8. The composition of claim 1, wherein the organic acid is present in an amount of from about 0.1% to 5.0% by weight.

9. The composition of claim 1, wherein the salt of the organic acid is present in amount of up to 7.5% by weight.

10. The composition of claim 1, wherein the water is present in an amount of from about 47.0% to 94.0% by weight.

11. The composition of claim 1, wherein the organic solvent is sodium lauryl ethoxysulfate.

12. The composition of claim 1, wherein the acid is an organic acid.

13. The composition of claim 9, wherein the organic acid is a hydroxy acid.

14. The composition of claim 9, wherein the acid is a citric acid.

15. The composition of claim 9, wherein the acid is hydroxy citric acid.

16. The composition of claim 11, wherein the citric acid is present in a concentration of about 0.1% to about 5% by weight.

17. The composition of claim 1, wherein the salt is a salt of an organic acid.

18. The composition of claim 1, wherein the sale is a salt of a hydroxy acid.

19. The composition of claim 1, wherein the salt is sodium citrate.

20. The composition of claim 15, wherein the sodium citrate is present in a concentration of about 0.1% to about 5% by weight.

21. The composition of claim 1, wherein the acid is citric acid and the salt is sodium citrate.

22. The composition of claim 19, wherein the amphoteric compound is cocamidopropyldimethyl betaine, the organic solvent is sodium lauryl ethoxysulfate, the acid is a citric acid and the salt is sodium citrate.

23. The composition of claim 22, wherein the cocamidopropyl betaine is present in an amount for from about 4.5% to about 25% by weight, the sodium lauryl ethoxysulfate is present in an amount of from about 0.5% to about 10.8% by weight, the citric acid is present in an amount from about 0.1% to about 5% by weight and the sodium citrate is present in an amount of from about 0.0 to about 3.0% by weight and the water is present in an amount from 81% to 10%.

24. The composition of claim 22 or 23, which further contains a biocide agent.

25. The composition of claim 22 or 23, which further contains an antimicrobial agent.

26. The composition of claim 22 or 23, which further contains a biocide agent and an antimicrobial agent, each of which is different from the other.

27. The composition of claim 20, wherein the biocide is selected from the group consisting of isothiazolinones.

28. The composition of claim 27, wherein the biocide is selected from the group consisting of 2-methyl-4-isothiazolin-3-one, 1,2 benzisothiazoline, and derivatives synthesized therefrom.

29. The composition of claim 1, which further contains triclosan.

30. The composition of claim 29, wherein the triclosan is present in an antimicrobial effective amount.

31. The composition of claim 1, which further contains ammonium bifluoride in an amount of at least 0.1% by weight.

32. The composition of claim 1, which further contains phosphoric acid in an amount of at least 0.2% by weight.

33. The composition of claim 1, which further contains ammonium bifluoride in an amount of at least 0.1% by weight and phosphoric acid in an amount of at least 0.2% by weight.

34. The composition of claim 32, which contains lauryl dimethylamine oxide.

35. The composition of claim 1, which further contains sodium tetraborate decahydrate.

36. The composition of claim 35, wherein the amount of sodium tetraborate decahydrate is in the range from about 4.0 to 16.0% by weight.

37. The composition of claim 1 which further contains an alkanol amine.

38. The composition of claim 36, wherein the alkanol amine is a monoalkanol amine.

39. The composition of claim 369, wherein the alkanol portion of the alkanol amine contains from 1 to 5 carbon atoms.

40. The composition of claim 19, wherein the alkanol amine is monoethanolamine.

41. The composition of claim 36, wherein the amount of the alkanol amine is from about 0.5 to 35. % by weight.

42. A composition comprising the following compounds:

| | |
|---|---|
| CAB | 15.0% |
| SLES | 6.75 |
| Citric ac. | 0.5 |
| Sod. Citrate | 0.75 |
| Biocide and/or | 0.5 |
| Antimicrobial | |
| Water | 76.50 |

43. A composition comprising the following compounds:

| | |
|---|---|
| CAB | 12.0% |
| SLES | 5.4% |
| Citric ac. | 0.4% |
| Amm. bifluoride | 2.4% |
| Phosphoric ac. | 5.0% |
| LDAO | 0.4% |
| Water | 74.4% |
| Triclosan | 2.2% |

44. A composition comprising the following compounds:

| | |
|---|---|
| CAB | 10.3% |
| SLES | 4.64% |
| Citric ac. | 0.34% |
| Sod. Citrate | 0.51% |
| Biocide and/or | 0.5% |
| Antimicrobial | |
| Borax decahydrate | 11.9% |
| MEA | 11.9% |
| Water | 59.91% |

45. A composition comprising the following compounds:

| | |
|---|---|
| CAB | 13.5% |
| SLES | 6.07% |
| Citric ac. | 1.45% |
| Sod. Citrate | 0.675% |
| Biocide and/or | 0.5% |
| Antimicrobial | |
| Water | 77.8% |

46. A method for removing noxious or malodorous components from material containing the same comprising treating the material with an anti-noxious or anti-malodorous effective amount of the composition of claim 1.

47. A method for removing noxious or malodorous components from material containing the same comprising treating the material with an anti-noxious or anti-malodorous effective amount of the composition of claim 23.

48. A method for removing noxious or malodorous components from material containing the same comprising treating the material with an anti-noxious or anti-malodorous effective amount of the composition of claim 42.

49. A method for removing noxious or malodorous components from material containing the same comprising treating the material with an anti-noxious or anti-malodorous effective amount of the composition of claim 43.

50. A method for removing noxious or malodorous components from material containing the same comprising treating the material with an anti-noxious or anti-malodorous effective amount of the composition of claim 44.

51. A method for removing noxious or malodorous components from material containing the same comprising treating the material with an anti-noxious or anti-malodorous effective amount of the composition of claim 45.

52. A method for removing noxious or malodorous components from material containing the same comprising treating the material with an anti-noxious or anti-malodorous effective amount of the composition of claim 43.

53. The method of any one of claims 46 through 52, wherein the material is selected from the group consisting of gases, solids, particulate solids and liquids.

54. The method of claim 52 wherein the microbes are in or on a gas, liquid or solid.

55. A method for making the composition of claim 42 comprising the steps of:
a. admixing the CAB and sodium lauryl ethics sulfate with gentle agitation;
b. separately mix the citric acid, sodium citrate, antimicrobial/biocide;
c. add the mixture from step b to that of step a with gentle agitation.

56. A method for making the composition of claim 44 comprising the steps of:
a. admixing the CAB and sodium lauryl ethics sulfate with gentle agitation;
b. heat the water to about 60° C. and add the borax decahydrate to the heated water to completely dissolve it;
c. separately mix the citric acid, sodium citrate, antimicrobial/biocide;
d. add the mixture from step c to the mixture from step b with gentle stirring;
e. add the mixture from step b to that of step d with gentle stirring;
f. add the monoethanolamine to the mixture of step e with gentle stirring.

57. A method for making the composition of claim 43 comprising the steps of:
a. admixing the CAB and sodium lauryl ethics sulfate with gentle agitation;
b. separately mix the citric acid, sodium citrate, antimicrobial/biocide;
c. mix ammonium bifluoride and water. When dissolved, admix in the phosphoric acid (85%) and lauryl dimethyl amine oxide;
d. add the mixture from step a to the mixture from step b with gentle mixing; the mixture will become thick and gel-like;
e. slowly add the mixture from step c to the mixture from step d with stirring.

58. A method for making the composition of claim 45 comprising the steps of:
a. admixing the CAB and sodium lauryl ethics sulfate with gentle agitation;
b. separately mix the citric acid, sodium citrate and water until the dry ingredients are dissolved;
c. add the mixture from step b to that of step a with gentle mixing.

59. A method for removing noxious or malodorous components from materials containing the same, the method comprising the steps of:
mixing a noxious or malodorous reducing composition with water, and
then spraying or fogging the composition into the atmosphere above the material to contact noxious or malodorous gases to provide an effective amount of the composition for removing noxious or malodorous components from materials containing the same.

60. The method of claim 59, wherein the effective amount of the noxious or malodorous reducing composition is at least about 0.002 grams per cubic meter of air.

61. The method of claim 59, wherein the malodorous gas is an acid gas.

62. The method of claim 59, wherein the malodorous gas is an alkaline gas.

63. The method of claim 59, wherein the malodorous gas is an aldehyde or ketone.

64. A method for controlling malodor in an internal poultry shed environment comprising spraying the composition of claim 44 or 45 into the atmosphere of the poultry shed to contact malodorous gases or applying the composition to malodor-producing materials to provide an effective amount of the composition.

65. A method for reducing noxious or malodorous or components from animal runs comprising; spraying the composition of claim 44 or 45 into the atmosphere over the animal run to contact malodorous gases or applying the composition to malodor-producing materials to provide an effective amount of the composition.

66. A method for controlling an external shed manure and odor comprising spraying the composition of claim 44 or 45 into the atmosphere of the shed to contact malodorous gases or applying the composition to malodor-producing materials to provide an effective; amount of the composition.

67. A method for controlling an internal pig shed environment comprising spraying the composition of claim 44 or 45 into the atmosphere over the shed to contact malodorous gases or applying the composition to malodor-producing materials to provide an effective amount of the composition.

68. A method for controlling an external pig shed environment comprising spraying the composition of claim 44 or 45 into the atmosphere over the shed to contact malodorous gases or applying the composition to malodor-producing materials to provide an effective amount of the composition.

69. A method for controlling cattle shed environment comprising spraying the composition of claim 44 or 45 into the atmosphere of the shed to contact malodorous gases or applying the composition to malodor-producing materials to provide an effective amount of the composition.

70. A method for controlling milking shed waste water comprising spraying the composition of claim 44 or 45 into the atmosphere over the pond to contact malodorous gases or applying the composition to malodor-producing materials to provide an effective amount of the composition.

71. A method for removing pollutants in environmental products comprising spraying the composition of claim 44 or 45 into the atmosphere over the products to contact malodorous gases or applying the composition to malodor-producing materials to provide an effective amount of the composition.

72. A method for removing pollutants in industrial products comprising spraying the composition of claim 44 or 45 into the atmosphere over the products to contact malodorous gases or applying the composition to malodor-producing materials to provide an effective amount of the composition.

73. A method for controlling industrial smokestack gas emissions comprising spraying the composition of claim 44 or 45 into the emissions to contact malodorous gases or applying the composition to malodor-producing materials to provide an effective amount of the composition.

74. A method for controlling emissions for an offal, rendering and meat processing plant comprising spraying the composition of claim 44 or 45 into the atmosphere to contact malodorous gases or applying the composition to malodor-producing materials to provide an effective amount of the composition.

75. A method for controlling odor of an external soak and oxidation pond of a pulp and paper mill treatment comprising spraying the composition of claim 44 and 45 into the atmosphere over the pond to contact malodorous gases or applying the composition to malodor-producing materials to provide an effective amount of the composition.

76. A method for controlling external processing ponds of a food and cannery plant comprising spraying the composition of claim 44 and 45 into the atmosphere over the pond to contact malodorous gases or applying the composition to malodor-producing materials to provide an effective amount of the composition.

77. A method for controlling odor emission of a food and cannery plant comprising spraying the composition of claim 44 and 45 into the emission to contact malodorous gases over the plant or applying the composition to malodor-producing materials to provide an effective amount of the composition.

78. A method for controlling emissions of a high volume pedestrian precincts comprising spraying the composition of claim 44 or 45 into the atmosphere over the precinct to contact malodorous gases or applying the composition to malodor-producing materials to provide an effective amount of the composition.

79. A method for controlling microbe and toxin removal comprising spraying, the composition of claim 43 into the atmosphere containing the microbes, germs or toxins to contact malodorous gases or applying the composition to malodor-producing materials to provide an effective amount of the composition.

80. A method for controlling air conditioning microbes comprising spraying the composition of claim 43 into the atmosphere of the air conditioner to contact malodorous gases or applying the composition to malodor-producing materials to provide an effective amount of the composition.

81. A method for controlling microbe and toxin removal comprising spraying the composition of claim 43 into the atmosphere to contact malodorous gases or applying the composition to malodor-producing materials to provide an effective amount of the composition.

82. A method for rendering fabrics microbe-free and odor-free comprising applying the composition of claim 43 to malodor-producing materials on the fabric to provide an effective amount of the composition.

83. A method for removing chemicals, microbes and particulate matter from filters comprising applying the composition of claim 43 to malodor-producing materials on the filters to provide an effective amount of the composition.

84. A method for removing microbes of food processing plants comprising spraying the composition of claim 43 into the atmosphere of the plant to contact malodorous gases or applying the composition to malodor-producing materials to provide an effective amount of the composition.

85. A method controlling border biosecurity comprising spraying the composition of claim 43 into the atmosphere of the border to contact malodorous gases or applying the composition to malodor-producing materials at the border to provide an effective; amount of the composition.

86. A method for removing microbes at airports and seaports comprising spraying the composition of claim 43 into the atmosphere to contact malodorous gases at the airport or seaport or applying the composition to malodor-producing materials to provide an effective amount of the composition.

87. A method for removing CO_{z}, H₂S and CO in subway air recirculation comprising spraying the composition of claim 43 into the recirculating air to provide an effective amount of the composition.

88. A method for removing microbes from the air-conditioning of public transportation vehicles comprising spraying the composition of claim 43 into the atmosphere of the vehicle to provide an effective amount of the composition.

89. A method for removing microbes from hospitals surfaces, equipment and facilities comprising spraying the composition of claim 43 into the atmosphere of the hospital to provide an effective amount of the composition.

90. A method for removing microbes, algae and odors from food processing plants comprising spraying the composition of claim 43 into the atmosphere to contact malodorous gases or applying the composition to malodor-producing materials to provide an effective amount of the composition.

91. A method for removing microbes and odors from port-a-loo comprising spraying the composition of claim 43 into the atmosphere to contact malodorous gases or applying the composition to malodor-producing materials to provide an effective amount of the composition.

92. A method for removing building mold comprising spraying the composition of claim 43 into the atmosphere above the mold to provide an effective amount of the composition.

93. A method for removing building mold and yeast comprising spraying the composition of claim 43 into the atmosphere to contact the yeast and mold to provide an effective amount of the composition.

94. A method for removing and preventing carpet mold comprising spraying the composition of claim 43 into the atmosphere to contact malodorous gases or applying the composition to malodor-producing materials to provide an effective amount of the composition.

95. A method for removing wallboard preparation mold comprising spraying the composition of claim 43 into the atmosphere to contact malodorous gases or applying the composition to malodor-producing materials to provide an effective amount of the composition.

96. A method for preventing and removing infection and microbes of medical and personal health care products comprising spraying the composition of claim 43 into the atmosphere above the products to provide an effective amount of the composition.

97. The method of claim 96, wherein the medical and personal health care products are tampons and sanitary pads.

98. A method for removing remove microbes and odors of age-care and living environment comprising spraying the composition of claim 43 into the atmosphere to provide an effective amount of the composition.

99. A method for removing microbes and odors of ostomy products comprising spraying the composition of claim 43 onto the ostomy products to provide an effective amount of the composition.

100. A method for removing harmful chemicals of anti-biological-chemical terrorist and warfare products comprising spraying the composition of claim 43 into the atmosphere to contact the chemicals to provide an effective amount of the composition.

101. A method for removing odors by using a consumer aerosol canister comprising spraying the composition of claim 44 or 45 into the atmosphere to contact malodorous gases or applying the composition to malodor-producing materials to provide an effective amount of the composition.

102. A method for removing harmful chemicals and microbes of a biological and chemical decontamination wash-down product comprising spraying the composition of claim 43 into the atmosphere to contact chemicals and microbes to provide an effective amount of the composition.

103. A method for removing microbes and odor of commercial, hospitality and dry cleaning products comprising spraying the composition of claim 43 into the atmosphere to contact malodorous gases or applying the composition to malodor-producing materials to provide an effective amount of the composition.

104. A method for removing odors by using a consumer aerosol canister comprising spraying the composition of claim 43 into the atmosphere to contact malodorous gases or applying the composition to malodor-producing materials to provide an effective amount of the composition.

105. A method for removing odors of a air purification unit comprising spraying the composition of claim 43 into the atmosphere to contact malodorous gases or applying the composition to malodor-producing materials to provide an effective amount of the composition.

106. A method for remove microbes and odors of prawn farms to comprising spraying the composition of claim 43 into the atmosphere to contact malodorous gases or applying the composition to malodor-producing materials to provide an effective amount of the composition.

107. A method for removing odors and soot of fires comprising spraying the composition of claim 44 or 45 into the atmosphere to contact malodorous gases or applying the composition to malodor-producing materials to provide an effective amount of the composition.

108. A method for removing or alleviating the odors caused by the fire and smoke comprising spraying the composition of claim 44 or 45 into the atmosphere to contact malodorous gases or applying the composition to malodor-producing materials to provide an effective amount of the composition.

109. The method of claim 108, wherein a fire detection system is programmed so that upon detection of a fire, an effective amount of the composition is released in the area of the fire and smoke.

110. A method for inhibiting or stopping the growth of yeast and mold fungi comprising spraying the composition of claim 43 into the atmosphere to contact the yeast and mold fungi to provide an effective amount of the composition.

111. A method for eliminating general pet odor comprising spraying the composition of claim 44 or 45 into the atmosphere to contact malodorous gases or applying the composition to malodor-producing materials to provide an effective amount of the composition.

112. A method for controlling bad breath comprising applying the composition of claim 44 or 45 to the oral cavity.

113. A method for eliminating organic household odors comprising spraying the composition of claim 44 or 45 into the atmosphere of the house to contact malodorous gases or applying the composition to malodor-producing materials to provide an effective amount of the composition.

114. An article of manufacture comprising the composition of claim 44 or 45 in a spray dispenser.

115. The article of manufacture of claim 114, wherein the spray dispenser is in fixed position in a room.

116. The article of manufacture of claim 114, wherein the spray dispenser is fitted adjacent to a toilet and the spray directed therein.

117. A method of claim 108 wherein an automatic fire door system incorporates a mechanism such that an effective amount of the composition is released on actuation of the door.

118. A smoke and fire detecting device comprising the spray dispenser of claim 114 activatingly connected to the detector such that when the detector is activated, the spray dispense is also activated to release the composition.

119. The composition of claim 1, which further comprises a fragrance.

120. The composition of claim 119, wherein the fragrance is present in an amount of about 1.5%.

121. The composition of claim 23, which further comprises a fragrance.

122. The composition of the claim 120, wherein the fragrance is present in an amount of about 1.5%.

123. The method of claim 46, wherein the composition further comprises a fragrance.

124. The composition of claim 123, wherein the fragrance is present in the amount of about 1.5%.

125. The method of claim 47, wherein the composition further comprises a fragrance.

126. The composition of claim 125, wherein the fragrance is present in the amount of about 1.5%.

127. The method of claim 48, wherein the composition further comprises a fragrance.

128. The composition of claim 127, wherein the fragrance is present in the amount of about 1.5%.
